(19) United States
(12) Patent Application Publication
(10) Pub. No.: US 2012/0225450 A1
(43) Pub. Date: Sep. 6, 2012

(54) CARBOXYL-CONTAINING POLYMERS WITH IMPROVED STORAGE STABILITY AND PROCESSABILITY

(75) Inventor: Frank-Peter Lang, Hattersheim (DE)

(73) Assignee: BASF SE, Ludwigshafen (DE)

(21) Appl. No.: 13/405,820

(22) Filed: Feb. 27, 2012

Related U.S. Application Data
(60) Provisional application No. 61/447,734, filed on Mar. 1, 2011.

(51) Int. Cl. C08L 33/08; C08K 3/36; C08K 5/098; C09K 8/02; C09D 11/10; C10M 145/14; C08K 3/34; C11D 3/37; D06M 15/19; D06L 3/00; D06P 1/00; D06L 1/00; C09K 3/00; G03F 7/32; C12P 1/00; B03D 1/016; C25D 15/00; C08L 33/02

(52) U.S. Cl. 435/41; 524/556; 524/560; 524/493; 524/492; 524/301; 507/119; 524/60; 508/469; 524/445; 510/276; 252/8.81; 252/8.83; 8/139; 8/147; 252/188.1; 8/636; 8/137; 430/331; 209/163; 205/109

(57) ABSTRACT

The present invention relates to carboxyl-containing polymers having higher storage stability and improved processability in the form of powders, pellets or granules and to their use in laundry detergents and cleaners, and also in further fields of use.

The present invention relates to carboxyl-containing polymers having higher storage stability and improved processability in the form of powders, pellets or granules and to their use in laundry detergents and cleaners, and also in further fields of use.

Carboxyl-containing polymers are chemical compounds comprising at least three carboxyl-containing monomer units. Examples are particularly polycarboxylic acids and their salts, the polycarboxylates.

Such carboxyl-containing polymers have long been an important constituent of state of the art, particularly phosphate-reduced or phosphate-free laundry detergents and cleaners for textiles. Their function is to control the so-called incrustation of textiles, meaning the deposition of insoluble, inorganic salts, for example calcium phosphate (in the case of phosphate-reduced laundry detergents), calcium carbonate, magnesium carbonate, calcium silicate or magnesium silicate (from the precipitation of the alkali donors sodium carbonate or sodium silicate with the hardness ions of water) or zeolites (an insoluble phosphate replacement) on the laundry. It is further necessary to prevent the deposition of these insolubles in the washing machine, particularly on the heating bars. Finally, carboxyl-containing polymers also counteract laundry graying, caused over many wash cycles by a homogeneous redeposition on the laundry from the wash liquor of small amounts of previously detached soil. Today, carboxyl-containing polymers are used in all types of laundry detergents, for example color detergents, heavy-duty detergents, mild detergents, specialty detergents for black textiles, detergents for jeans, and wool detergents.

The use concentration is generally between 0.5% and 5.0% by weight, particularly between 1% and 4% by weight and especially between 1.5% and 3% by weight of carboxyl-containing polymer based on the laundry detergent. The laundry detergents in which carboxyl-containing polymers are used can be in solid form, for example in the form of powders, granules, pellets, tablets, bar soaps, in semi-liquid form, for example in the form of gels or pastes, or in liquid form.

Carboxyl-containing polymers are also used as process aids in the production of washing powders by the hot-spray process. In this process, a slurry of laundry detergent having a very high solids content is sprayed into a spray tower and the water is evaporated by countercurrent hot air. Here, the carboxyl-containing polymers reduce slurry viscosity and permit higher solids and lower water contents, and thus reduce the energy requirements of the spray-drying operation.

A further very important field of use for carboxyl-containing polymers is in dishwasher detergents, which can be formulated to be phosphate containing or phosphate free. Here the polymers augment the cleaning effect on crockery (porcelain, stoneware, earthenware, plastics, for example polypropylene or polyethylene, thermosets, for example melamine), on cutlery (for example made of metals, such as stainless steel, silver and also plastics) and also on pots, pans, etc., via their excellent soil-dispersing ability, and prevent the deposition of unsightly deposits, of calcium and magnesium salts for example, on the ware to be cleaned and on machine parts.

Carboxyl-containing polymers are further used in many cleaners for trade, industry and home, for cleaning surfaces made of glass, porcelain, stoneware, earthenware, natural stone, coated surfaces of metals and plastics, uncoated metals and plastics, synthetic resins, linoleum, wood and also manufactured and natural fibers (carpets for example). The cleaners may be adjusted to an acidic, neutral or alkaline pH.

Examples of cleaners utilizing carboxyl-containing polymers are all-purpose cleaners, car shampoos, rim cleaners, low temperature cleaners, disinfecting cleaners, dairy cleaners, sanitary cleaners, carpet cleaners, cleaners in metal pretreatment for cleaning of iron, steel, zinc, tin, aluminum, copper, brass and other metals. Again, the dispersing ability of these polymers plays an important part in stabilizing the detached soil in the wash liquor and in avoiding deposits of alkaline earth metal and heavy metal phosphates, carbonates, silicates, soaps and also of insoluble salts of anionic surfactants on apparatus parts and the surfaces to be cleaned.

Water treatment is another important field of use. Water is used in many industrial and chemical processes, for example as cooling water, boiler feed water, process water and extraction water. A key problem here is process disruption due to the introduction of unwanted metal ions, for example the Ca²⁺ and Mg²⁺ hardness ions of water, and heavy metal ions. These are responsible for the formation of sparingly soluble deposits of alkaline earth metal or heavy metal salts. Consequences include, for example, deposits of calcium sulfate, calcium carbonate, calcium phosphate or magnetite in the steam generators of thermal power plants, boilers, evaporation plants, heating circuits and on heat exchanger surfaces and hence an impairment of heat transition and also very generally the plugging of lines, filters, valves or nozzles. Carboxyl-containing polymers prevent the formation of deposits and crystal growth and keep the insoluble deposits suspended in a finely dispersed state. Water treatment as a field also includes seawater desalination, which can take place thermally via a distillation or via reverse osmosis.

Further applications for carboxyl-containing polymers are the use in fermentation processes, for example for producing alcohol, in metal processing for producing emulsion and solution concentrates, for example lubricants and impregnants, drill oils, drawing oil emulsions, water-thinnable and water-soluble metal processing auxiliaries, hydraulic solutions and emulsions and to avoid the precipitation of calcium salts of fatty acids and their dispersal in aqueous cooling lubricants; as grinding auxiliaries for the comminution of solids which later have to be dispersed, for example organic color pigments, titanium dioxide pigments, iron oxide pigments and carbon black pigments; in the manufacture of printing inks; to stabilize crop protection agent dispersions; as protective colloids in rubber production by solvent polymerization; to produce coal-water slurries; in the paper industry as dispersants for fillers such as calcium carbonate or kaolin; to stabilize and superplasticize ceramic slips; in the mining of ores for floatation concentration in ore beneficiation; in surface technology, for example in electrocoating baths to improve the stability of the baths and to optimize the reactions taking place at the anode and the cathode; in textile finishing in processing and finishing operations such as scouring, kiering, bleaching, dyeing, washing and resin finishing of the fabrics and as a constituent of sizing agents; in leather manufacture for vegetable tanning particularly in the case of iron-containing water; in photographic baths (developer baths, bleaching baths and bleach-fixing baths).

Polycarboxylic acids as an important group of the carboxyl-containing polymers are obtained by homo- or copolymerization of vinylic carboxyl-containing monomers such as, for example, acrylic acid, methacrylic acid, maleic acid, fumaric acid, itaconic acid, citraconic acid, mesaconic acid, aconitic acid and/or methylenemalonic acid. These can additionally be reacted as comonomers in the form of their esters and/or in the form of their amides with the free carboxylic acid monomers. The carboxyl-containing polymers may further comprise carboxyl-free comonomers.

To use the carboxyl-containing polymers in laundry detergent and cleaner compositions, as well as in the other abovementioned fields of use, they are on offer as aqueous solutions or in solid form, as powders or granules.

Aqueous solutions of carboxyl-containing polymers have the disadvantage of only being obtainable with relatively low active-ingredient contents, which increases the cost of transportation to the customer. They further require heatable tanks for storage and corresponding systems to feed them to the use territory (the water treatment for example) or the manufacturing operation (for laundry detergent and cleaner formulations for example), for example pumps, pipework and nozzles, which represents an appreciable capital expense.

The powder-, pellet- and granule-shaped carboxyl-containing polymers that are commercially available in turn have the disadvantage of being very hygroscopic. This greatly inconveniences their handling, particularly in climatic zones having a comparatively high humidity. Therefore, it is often the case that special measures, such as controlled climatic conditions in manufacturing facilities for example, have to be taken, which again entails an appreciable capital expense. Moreover, opened containers have to be carefully resealed, which can be a problem with Big Bags. Irrespective of their hygroscopicity, particularly the pulverulent carboxyl-containing polymers exhibit high forces of cohesion between the particles and hence also very poor flow performance, which appreciably inconveniences conveying and exact metering. Finally, the products exhibit poor storage stability when exposed to the mechanical impact of pressure which acts on the packaged products in the stacking of Big Bags for example and which can lead to caking.

It is an object of the present invention to provide a system in which the carboxyl-containing polymers are present in the form of powders, granules or pellets, these have good physical stability and are simple to store and process by the user.

We have found that this object is achieved, surprisingly, when certain finely divided additives are added to the carboxyl-containing polymers which are present in solid form.

The present invention accordingly provides a mixture comprising powder-, granule- or pellet-shaped carboxyl-containing polymer and finely divided additive.

For the purposes of this invention, powder-shaped materials have a particle size in the range from 1 μm to 0.1 mm, granule-shaped materials have a particle size in the range from 0.1 mm to 2 mm and pellet-shaped particles have a particle size in the range from 2 mm to 5 mm.

For the purposes of this invention, by “finely divided” in reference to the additive is meant a particle size in the range from 1 to 500 μm.

Mixtures comprising one or more different carboxyl-containing polymers and one or more finely divided additives may be concerned. A mixture comprising a carboxyl-containing polymer in powder, pellet and/or granule form and a finely divided additive is a particularly preferred embodiment. A mixture consisting of a carboxyl-containing polymer in powder, pellet and/or granule form and a finely divided additive is a particularly preferred embodiment.

This particularly preferred embodiment will now be described, but all other embodiments, i.e., with multiple polymers and/or multiple additives, are similarly improvable via further features and hence also described mutatis mutandis.

The mixture of the present invention comprises the carboxyl-containing polymer in solid form, i.e., in the form of powders, granules or pellets, in an amount of 90% to 99.99% by weight, and also 0.01% to 10% by weight of a finely divided additive, the weight % ages being based on the total amount of the two constituents.

Polycarboxylic acids as an important group of the carboxyl-containing polymers are obtained by homo- or copolymerization of vinylic carboxyl-containing monomers such as, for example, acrylic acid, methacrylic acid, maleic acid, fumaric acid, itaconic acid, citraconic acid, mesaconic acid, aconitic acid and/or methylenemalonic acid. These can be additionally reacted as comonomers in the form of their esters (for example esterified with primary, secondary or tertiary alcohols R—OH where R═C1- to C18-alkyl, benzyl, ethylphenyl, cyclohexyl or phenyl, preferably with methanol, ethanol or propanol) and/or in the form of their amides (for example amidated with ammonia; primary or secondary amines, R—NH₂ and R₁—NH—R₂, where R, R₁ and R₂=C1- to C18-alkyl, benzyl, ethylphenyl, cyclohexyl or phenyl) with the free carboxylic acid monomers.

The carboxyl-containing polymers may further comprise carboxyl-free comonomers. Examples thereof are C2- to C30-olefins, preferably C2- to C24-olefins, more preferably C2- to C18-olefins and most preferably C2- to C8-olefins (ethylene, propylene and butylene, diisobutene); vinyl acetate; vinyl alcohol; styrene; vinyl methyl ether; vinylcyclohexane; vinyl chloride; acrylonitrile; N-vinylimidazoline, N-vinylpyrrolidone, N-vinylpyridine, diallyldimethylammonium chloride, N-vinylformamide, N-vinylacetamide, vinylamine, allylamine.

Carboxyl-containing polymers are commercially available as free polycarboxylic acids, in partially neutralized form or in salt form, fully neutralized as polycarboxylates. In partially or fully neutralized form, they are usually used as sodium salts. In principle, however, other cations, such as lithium; potassium, ammonium; substituted ammonium such as mono-, di-, trialkylammonium, with C₁- to C₁₈-alkyl or mono-, di- and trialkanolammonium, for example triethanolammonium, can also be present. The polymers are present with pH values starting with 1 for the acid form and going up to 14 for the fully neutralized salt form. The molar masses of the polycarboxylic acids and their salts is between about 1000 g/mol (e.g., Sokalan® PA 13 PN) up to about 250 000 g/mol (e.g., Sokalan® PA110 S). Accordingly, the K values of the polymers are between about 10 to about 110. The K-value of a polymer provides information about the intrinsic viscosity of the polymer and hence is a further parameter to characterize the degree of polymerization and the molar mass. The K-values of 1% aqueous solutions of the polymers are determined at pH 7.

Examples of carboxyl-containing polymers are for example polyacrylic acid and its salts, the polyacrylates (e.g., Sokalan® PA 25 CL Granulat, Sokalan® PA 30 CL Granulat, Sokalan® PA 30 CL PN Granulat, Sokalan® PA 40 Pulver from BASF); polymaleic acid (e.g., Belgard® or Belsperse® from BWA); copolymers of acrylic acid and maleic acid (e.g., Sokalan® CP 5 Granulat, Sokalan® CP 45 Granulat, Sokalan® CP 7 Granulat NL from BASF), copolymers and terpolymers based on acrylic acid and/or maleic acid and a non-carboxyl-containing monomer (e.g., Sokalan® CP 35, Sokalan® CP 42, Sokalan® CP 50, Sokalan® PM 10 I from BASF).

Polymers that differ in the monomers used or in the ratio between the monomers used can also be used as a mixture in the present invention mixtures of carboxyl-containing polymer and additive.

Suitable additives are for example fatty acids, particularly C₁₆-C₂₂ fatty acids, such as tallow fatty acid, stearic acid, behenic acid and salts thereof, preferably alkaline earth metal ions, more preferably with Ca²⁺ and Mg²⁺; fatty alcohols; cellulose; waxes, for example montan waxes, paraffin waxes, ester waxes and polyolefin waxes; magnesium oxide; kaolin; talc, tricalcium phosphate and silicas.

The aforementioned additives can be used not only individually but also in admixture in the mixture of the present invention, comprising carboxyl-containing polymer and additive.

Preferred additives include the Ca²⁺ and Mg²⁺ salts of C₁₆-C₂₂ fatty acids, especially calcium stearate and magnesium stearate. Particularly preferred additives are synthetic, colloidal, pyrogenous silicas or synthetic, colloidal, precipitated silicas. Pyrogenous silicas are obtained by high-temperature flame hydrolysis of silicon tetrachloride in the detonating gas flame. Precipitated silicas are obtained wet-chemically from alkali metal silicate solutions by addition of acids. Pyrogenous silicas as well as precipitated silicas are amorphous in structure, not crystalline.

Examples of pyrogenous silicas are the Aerosil brands (from Evonik), particularly Aerosil® 200, and examples of the precipitated silicas are the Sipernat brands (from Evonik), particularly Sipernat® 320, Sipernat® 320 DS, Sipernat® 360, Sipernat® 500 LS, Sipernat® 2200, Sipernat® 22, Sipernat® 22 S, Sipernat® 22 LS, Sipernat® 50, Sipernat® 50 S, Sipernat® C 600, Sipernat® C 630, Sipernat® 820 A and Sipernat® 880. The present invention mixtures of carboxyl-containing polymer and additive can utilize not only the aforementioned hydrophilic silicas but also hydrophobically modified silicas. Hydrophobic silicas are for example Sipernat® D 10, Sipernat® D 17 and Aerosil® R 812 and R 972. Hydrophobically modified, colloidal, synthetic precipitated silicas and hydrophobically modified, colloidal, pyrogenously produced silicas are very particularly preferred additives.

Colloidal, synthetic precipitated silicas are characterized by a high specific surface area of 30-500 m²/g, preferably 150-450 m²/g. Hydrophobically modified precipitated silicas have a high specific surface area of preferably 75-125 m²/g. (Determination as per area meter method ISO 5794-1 Annex D). The tamped density is 50-300 g/L, preferably 75-200 g/L and even more preferably 90-150 g/L. (Determined to DIN ISO 787/11, after tamping under defined conditions). The colloidal pyrogenous silicas preferably have specific surface areas of 100-400 m²/g and particle sizes of 1 nm-50 nm. Tamped density is about 50-150 g/L.

The synthetic silicas described, in contradistinction to the silicates used in laundry detergents, comprise metal ions such as Ca²⁺, Mg²⁺, Al³⁺ or Fe³⁺ in very low amounts, i.e., <5% by weight, preferably less than 3% by weight, more preferably less than 2% by weight and more preferably less than 1.5% by weight, based on the total weight of the particular synthetic silica.

These do not constitute an effective constituent part of the chemical compound, but are impurities introduced by the nature of the manufacturing operation. For instance, the sodium content determined as Na₂O (ISO 3262-18) is <1.5% by weight. Crystalline silicates, sheet-silicates and zeolites do not comprise part of the preferred additives.

The present invention mixtures comprising the carboxyl-containing polymer comprise the finely divided additive in a concentration of 0.01% to 10% by weight, preferably 0.1% to 5% by weight, more preferably 0.5% to 4% by weight and most preferably 1% to 3% by weight.

The mixtures of the present invention are produced by mixing the carboxyl-containing polymer, present as a powder, pellet or granules, with the finely divided additive.

The typical particle size of the carboxyl-containing polymer is in the range from 10 to 2500 μm, preferably in the range from 20 to 1500 μm, more preferably in the range from 50 to 1000 μm and most preferably in the range from 100 to 700 μm.

By “finely divided” in reference to the additive is meant a particle size in the range from 1 to 500 μm.

In the case of the colloidal pyrogenous silicas the additives preferably have a particle size in the range from 1 to 50 nm. In the case of the precipitated silicas the additives preferably have a particle size in the range from 1 to 200 μm preferably in the range from 5 to 150 μm and more preferably in the range from 8 to 120 μm. Each determined by light scattering as per ISO13320-1.

The mixing operation can be carried out in the customary mixing assemblies, for example drum mixers, V-blenders, tumble or Turbula mixers, cone mixers (e.g., Nauta mixers), plowshare mixers (Lodige mixer, Eirich mixer). In one preferred embodiment, the mixing operation takes place in mixers that exert low shearing forces on the material being mixed, for example tumble mixers, cone mixers and plowshare mixers.

Typically, the carboxyl-containing polymer is initially charged, then the additive is added and this is followed by mixing. In order to ensure gentle mixing, the shortest possible mixing times are employed. For example, a mixing time of 3 minutes is completely sufficient to produce 100 g of the mixture of the present invention in a Turbula mixer.

When the carboxyl-containing polymers in powder form are produced via spray drying, the additive is advantageously metered directly to the spray tower separately from the aqueous solution of the polymer. Possible points for addition are the upper end of the spray dryer, a metering terminal via a sight glass or the metered addition via the hot air stream. The same holds in principle for spray granulation.

When metered addition of the additive into the spray tower is not possible, it can be added in a separate mixing assembly, as previously described.

Should the additive to be admixed to the carboxyl-containing polymer not be sufficiently finely divided because, for example, it is only available as a material which has been melted by heating and has resolidified after filling into drums, as may be the case with fatty acids, fatty alcohols or waxes for example, the carboxyl-containing polymer can be ground together with the additive, likewise ensuring commixing of the components. Any grinding apparatus is suitable for this in principle, such as impact mills and cutting mills for example.

The use concentration of the present invention mixture comprising carboxyl-containing polymer and additive in laundry detergents and cleaners depends on the concentration of the carboxyl-containing polymer present in the mixture. Based on the polymer (without additive) the use concentration in the laundry detergent is between 0.5% and 5.0% by weight, preferably between 1% and 4% by weight and most preferably between 1.5% and 3% by weight.

The laundry detergent and cleaner formulations in which the present invention mixtures comprising carboxyl-containing polymer and additive can be used are preferably solid laundry detergents in the form of powders, granules, pellets, tablets or bar soaps. However, they can also be incorporated into liquid laundry detergents, laundering gels and laundering pastes. This will preferably be the case when long transists to the user make it desirable to supply the carboxyl-containing polymer in a highly concentrated form and hence not as an aqueous solution, and the protection of the product from caking due to moisture absorption in the course of transportation (on a ship for example) is to be improved.

Laundry detergents and cleaners comprising the present invention mixture comprising carboxyl-containing polymer and additive can further comprise further customary constituents. These will now be described:

### Anionic Surfactants

The anionic surfactants used are preferably alkylbenzenesulfonates, alkanesulfonates, olefinsulfonates, alkyl ester sulfonates, alkyl sulfates, alkyl ether sulfates alkyl carboxylates (soaps) and alkyl phosphates. The counter-ions present are alkali metal cations, preferably sodium or potassium, alkaline earth metal cations, for example calcium or magnesium, and also ammonium NH₄⁺ and substituted ammonium compounds, for example mono-, di- or triethanolammonium cations, and mixtures of the aforementioned cations therefrom.

Alkenyl- or alkylbenzenesulfonates may comprise a branched or linear, optionally hydroxyl-substituted alkenyl or alkyl group. Preferably they comprise linear alkyl chains having 9 to 25 carbon atoms and more preferably having 10 to about 13 carbon atoms.

Alkanesulfonates are available on a large industrial scale in the form of secondary alkanesulfonates wherein the sulfo group is attached to a secondary carbon atom of the alkyl moiety. The alkyl group can in principle be saturated, unsaturated, branched or linear and optionally hydroxyl substituted. Preferred secondary alkanesulfonates comprise linear C₉ to C₂₅-alkyl radicals, preferably C₁₀ to C₂₀-alkyl radicals and more preferably C₁₃ to C₁₇-alkyl radicals. Olefinsulfonates are obtained by sulfonation of C₈ to C₂₄— and preferably C₁₄ to C₁₆-α-olefins with sulfur trioxide and subsequent neutralization. Owing to their production process, these olefinsulfonates may comprise minor amounts of hydroxyalkanesulfonates and alkanedisulfonates.

Alkyl ester sulfonates derive for example from linear esters of C₈ to C₂₀-carboxylic acids, i.e., fatty acids, which are sulfonated with sulfur trioxide. The fatty acids are obtained from natural fats, such as tallow, coconut oil and palm oil for example, or can be synthetic in nature.

### Compounds of Formula (I)

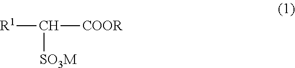

are preferred for laundry detergent and cleaner applications.

In formula (I), R¹ is a C₈ to C₂₀-alkyl radical, preferably C₁₀ to C₁₆-alkyl and R is a C₁ to C₆-alkyl radical, preferably a methyl, ethyl or isopropyl group. Particular preference is given to methyl ester sulfonates where R¹ is C₁₀ to C₁₆-alkyl.

Alkyl sulfates are surfactants of the formula R²OSO₃M, where R² is C₁₀-C₂₄-alkyl and preferably C₁₂-C₁₈-alkyl. M is a counter-ion as described at the beginning for anionic surfactants.

Alkyl ether sulfates have the general structure R²O(A)ₘSO₃M, where R² is a C₁₀-C₂₄-alkyl and preferably C₁₂-C₁₈ alkyl radical.

A is an alkoxy unit, preferably ethoxy and m is a value from about 0.5 to about 6, preferably between about 1 and about 3, and M is a cation, for example sodium, potassium, calcium, magnesium, ammonium or a substituted ammonium cation.

Alkyl carboxylates are generally known by the term “soap”. Soaps can be manufactured on the basis of saturated or unsaturated, preferably native, linear C₈ to C₁₈-fatty acids.

Further anionic surfactants are salts of acylamino carboxylic acids, acyl sarcosinates, fatty acid-protein condensation products obtained by reaction of fatty acid chlorides with oligopeptides; salts of alkylsulfamido carboxylic acids; salts of alkyl and alkylaryl ether carboxylic acids; sulfonated polycarboxylic acids, alkyl and alkenyl glycerol sulfates such as oleyl glycerol sulfates, alkylphenol ether sulfates, alkyl phosphates, alkyl ether phosphates, isethionates, such as acyl isethionates, N-acyltaurides, alkyl succinates, sulfosuccinates, monoesters of sulfosuccinates (particularly saturated and unsaturated C₁₂-C₁₈-monoesters) and diesters of sulfosuccinates (particularly saturated and unsaturated C₁₂-C₁₈-diesters), sulfates of alkylpolysaccharides such as sulfates of alkylpolyglycosides and alkyl polyethoxy carboxylates such as those of the formula RO(CH₂CH₂)ₖCH₂COO⁻M⁺, where R is C₈ to C₂₂-alkyl, k is a number from 0 to 10 and M is a cation.

### Nonionic Surfactants

A distinction is made according to the type of the hydrophobic and the hydrophilic base:

Condensation products of alcohols with ethylene oxide. The alcohols have a C₈ to C₂₂-alkyl group, preferably a C₁₀ to C₁₈-alkyl group, which may be linear or branched, primary or secondary. These are condensed with about 1 mol to about 25 mol and preferably with about 3 mol to about 18 mol of ethylene oxide per mole of alcohol. Examples of commercially available nonionic surfactants of this type are the Lutensol brands (BASF), the Empilan brands (Huntsman) and the Genapol brands (Clariant).

Condensation products of alcohols with ethylene oxide and a further alkylene oxide. These can be constructed according to the scheme R³—O-EO-AO or R³—O-AO-EO, where R is a primary or secondary, branched or linear C₈ to C₂₂-alkyl group, preferably a C₁₀ to C₁₈-alkyl group, EO is ethylene oxide and AO comprises an alkylene oxide, preferably propylene oxide, butylene oxide or pentylene oxide. Known products are the Plurafac LF brands (BASF).

Condensation Products of Polypropylene Glycol with Ethylene Oxide.

The hydrophobic moiety of these compounds preferably has a molecular weight between about 1500 and about 1800. The addition of up to about 40 mol of ethylene oxide onto this hydrophobic moiety leads to amphiphilic compounds. Commercially available examples of this class of products are the Pluronic® brands from BASF and the Genapol® PF brands from Clariant GmbH.

Condensation Products of Ethylene Oxide with a Reaction Product of Propylene Oxide and Ethylenediamine.

The hydrophobic unit of these compounds consists of the reaction product of ethylenediamine and propylene oxide and generally has a molecular weight of about 2500 to 3000. It is onto this hydrophobic unit that ethylene oxide is added up to a content of about 40% to about 80% by weight of polyoxyethylene and a molecular weight of about 5000 to 11 000. Commercially available examples of this class of compounds are the Tetronic® brands (BASF) and the Genapol® PN brands (Clariant).

### Fatty Acid Amides

Fatty acid amides have the general formula

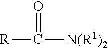

where R is an alkyl radical having 7 to 21 and preferably 9 to 17 carbon atoms.

The two R¹ alkyl radicals may be the same or different and may independently be hydrogen, C₁-C₄-alkyl, C₁-C₄-hydroxyalkyl or (C₂H₄O)ₓH, where x varies from 1 to 3. Preference is given to C₈-C₂₀-amides as monoethanolamides, diethanolamides and diisopropanolamides.

### Semipolar Nonionic Surfactants

Nonionic surfactants also include water-soluble amine oxides, water-soluble phosphine oxides and water-soluble sulfoxides, having at least one C8 to C18-alkyl radical, preferably a C10 to C14-alkyl radical which confers the amphiphilic character on the compound. Preferably C₁₀-C₁₈-alkyldimethylamine oxides and C₈-C₁₂-alkoxyethyldihydroxyethylamine oxides are used in laundry detergents and cleaners.

Useful nonionic surfactants further include alkyl and alkenyl oligoglycosides and also fatty acid polyglycol esters or fatty amine polyglycol esters each having 8 to 20 and preferably 12 to 18 carbon atoms in the fatty alkyl moiety, and fatty acid N-alkylglucamides.

### Zwitterionic Surfactants

Typical examples of amphoteric or zwitterionic surfactants are alkylbetaines, alkylamide betaines, aminopropionates, aminoglycinates and amphoteric imidazolinium compounds.

Particular preference is given to N-alkyl-N,N-dimethyl-N-carboxymethylbetaines and also N-(alkylamidopropyl)-N,N-dimethyl-N-carboxymethylbetaines, and also alkyldipolyethoxybetaines, each with a linear or branched alkyl radical of 8 to 22 carbon atoms, preferably 8 to 18 carbon atoms and more preferably having about 12 to about 18 carbon atoms.

### Cationic Surfactants

These are substituted or unsubstituted straight-chain or branched quaternary ammonium salts of the type R¹N(CH₃)₃⁺X⁻, R¹R²N(CH₃)₂⁺X⁻, R¹R²R³N(CH₃)⁺X⁻ or R¹R²R³R⁴N⁺X⁻. The R¹, R², R³ and R⁴ radicals may preferably be independently unsubstituted alkyl having a chain length between 8 and 24 carbon atoms and more particularly between 10 and 18 carbon atoms, hydroxyalkyl having about 1 to about 4 carbon atoms, phenyl, C₂- to C₁₈-alkenyl, C₇- to C₂₄-aralkyl, (C₂H₄O)ₓH, where x is from about 1 to about 3, alkyl radicals comprising one or more ester groups, or cyclic quaternary ammonium salts. X is a suitable anion.

### Builders

Builders can be present in the laundry detergent and cleaner compositions at weight fractions of about 5% to about 80%. Builders comprise for example alkali metal, ammonium and alkanolammonium salts of polyphosphates such as, for example, tripolyphosphates, pyrophosphates, glassy polymeric metaphosphates, orthophosphates, phosphonates, silicates, carbonates including bicarbonates and sesquicarbonates, sulfates and aluminosilicates.

Aluminosilicate builders are preferred for the present invention. Particular preference is given to zeolites of the formula Na_{z}[(AlO₂)_{z}(SiO₂)_{y}].xH₂O, where z and y are integers of at least 6, the ratio of z to y is between 1.0 to about 0.5, and x is an integer from about 15 to about 264.

These aluminosilicates can be crystalline or amorphous in structure, and can be naturally occurring or else synthetically produced. Processes for preparing ion exchangers based on aluminosilicate are described in U.S. Pat. No. 3,985,669 and U.S. Pat. No. 4,605,509. Particular preference is given to synthetic crystalline aluminosilicates, such as zeolite A, zeolite P(B) and zeolite X.

Alkali metal silicates, particularly those having an SiO₂:Na₂O ratio between 1.6:1 and 3.2:1 and also sheet-silicates, for example sodium sheet-silicates as described in U.S. Pat. No. 4,664,839, e.g., SKS 6® (Clariant).

### Bleaching Agents

The laundry detergent and cleaner compositions comprising the present invention mixtures of carboxyl-containing polymer and additive may further comprise one or more bleaching agents and also bleach activators, bleach catalysts and suitable stabilizers. The bleaching agents used are persalts such as perborates (perborate monohydrate, perborate tetrahydrate) and percarbonate. Persalts are generally combined with so-called bleach activators, preferably tetraacetylethylenediamine. Bleach activators are often referred to in the prior art as organic peroxyacid precursors since they react with the persalt to liberate a peracid such as peracetic acid for example. Depending on the stability of the peracid in question, its physical state and the form of laundry detergent, it can also be used directly in the laundry detergent. Examples of peroxyacids preferred for use in this invention include peroxydodecanedioic acid (DPDA), the nonylamide of peroxysuccinic acid (NAPSA), the nonylamide of peroxyadipic acid (NAPAA) and decyldiperoxysuccinic acid (DDPSA), nonanoylamidocaproyloxybenzenesulfonic acid and alkanoyloxybenzenesulfonic acids such as nonanoyloxybenzenesulfonic acid (NOBS) and lauroyloxybenzenesulfonic acid (LOBS). Particular preference is given to using bleach systems based on perborate and/or percarbonate with the bleach activator tetraacetylethylenediamine (TAED) in the laundry detergent and cleaner compositions of the present invention.

The laundry detergent and cleaner compositions comprising the present invention mixtures comprising carboxyl-containing polymer and additive may further comprise the customary assistants which boost the cleaning performance, serve to recondition the textile to be washed, or modify the performance characteristics of the laundry detergent composition. Suitable auxiliaries include for example enzymes, more particularly proteases, lipases, cellulases and amylases, mannanases, glycosidases, enzyme stabilizers, foam boosters, foam retarders, anti-tarnish and/or -corrosion agents, suspendants, dyes, fillers, optical brighteners, disinfectants, complexing agents, alkalis, hydrotropic compounds, antioxidants, perfumes, solvents, solubilizers, soil antiredeposition agents, dispersants, processing aids, plasticizers, softeners and antistats.

The present invention further provides for the use of the present invention mixture in textile auxiliaries, in fermentation processes, in metal processing, drill oils, drawing oil emulsions, water-thinnable and water-soluble metal processing auxiliaries, hydraulic solutions and emulsions and to avoid the precipitation of calcium salts of fatty acids and their dispersal in aqueous cooling lubricants; as grinding auxiliaries for the comminution of solids, in the manufacture of printing inks; to stabilize crop protection agent dispersions; as protective colloids in rubber production, to produce coal-water slurries, in the paper industry as dispersants for fillers such as calcium carbonate or kaolin; to stabilize and superplasticize ceramic slips; in the mining of ores for floatation concentration in ore beneficiation; in surface technology, to improve the stability of electrocoating baths and to optimize the reactions taking place at the anode and the cathode; in textile finishing in processing and finishing operations during the scouring, kiering, bleaching, dyeing, washing and resin finishing of fabrics and as a constituent of sizing agents; in photographic baths, developer baths, bleaching baths and bleach-fixing baths.

### EXAMPLES

### Example 1

Mixtures of Sokalan® CP 42 (=modified polyacrylic acid, sodium salt) and in each case 2% by weight and 5% by weight of the hydrophobically modified silica Sipernat® D 10 were prepared. To this end, the requisite amounts of the two components were gently mixed in a container for 3 min using a tumble mixer. To investigate storage stability, the mixtures were stored in a Petri dish in a conditioning cabinet at 33° C./65% relative humidity and visually assessed for 7 days. For comparison, Sokalan® CP 42 was stored without added silica.

TABLE 1
storage test of Sokalan ® CP 42 (=modified polyacrylic acid sodium salt) with and
without addition of Sipernat ® D 10. Visual assessment at 33° C./65% relative humidity.

| | Visual assessment at 33° C./65% relative humidity | | | | | |
|---|---|---|---|---|---|---|
| Sokalan ® CP 42 | 0 h | 8 h | 24 h | 2 days | 3 days | 1 week |
| a) no additive | badly | surface | Homo- | almost | — | — |
| | flowable | pasty and | geneous | clear, | | |
| | granules | tacky | cloudy, | yellowish, | | |
| | | | pasty | tacky mass | | |
| | | | mass | | | |
| b) with 2% of | readily | flowable | granules | granules | granules | granules partly |
| Sipernat ® D 10 | flowable | granules | partly | begin to | partly | sintered, |
| | granules | | adhered | sinter | sintered, | <50% |
| | | | | | >50% | flowable |
| | | | | | flowable | |
| c) with 5% of | readily | flowable | granules | granules | granules | granules partly |
| Sipernat ® D 10 | flowable | granules | partly | begin to | partly | sintered, |
| | granules | | adhered | sinter | sintered, | >50% |
| | | | | | <50% | flowable |
| | | | | | flowable | |

### Example 2

Mixtures of Sokalan® CP 50 (=modified polyacrylate) and in each case 2% by weight and 5% by weight of the hydrophobically modified silica Sipernat® D 17 were prepared. To this end, the requisite amounts of the two components were gently mixed in a container for 2 min using a tumble mixer. To investigate storage stability, the mixtures were stored in a Petri dish in a conditioning cabinet at 33° C./65% relative humidity and visually assessed for 7 days. For comparison, Sokalan® CP 50 was stored without added silica.

TABLE 2
storage test of Sokalan ® CP 50 (=modified polyacrylate) with and without
addition of Sipernat ® D 17. Visual assessment at 33° C./65% relative humidity.

| | Visual assessment at 33° C./65% relative humidity | | | | | |
|---|---|---|---|---|---|---|
| Sokalan ®CP 50 | 0 h | 4h | 8 h | 24 h | 2 days | 7 days |
| a) no additive | readily | granules | partially | granules | tacky mass, | — |
| | flowable | slightly | pasty | mostly | storage test | |
| | granules | adhered | mass | sintered | terminated | |
| b) with 2% of | Readily | flowable | flowable | granules | granules | granules |
| Sipernat ® D 17 | flowable | granules | granules | swollen | swollen but | swollen but |
| | granules | | | but | flowable | flowable |
| | | | | flowable | | |
| c) with 5% of | Readily | flowable | flowable | granules | granules | granules |
| Sipernat ® D 17 | flowable | granules | granules | swollen | swollen but | swollen but |
| | granules | | | but | flowable | flowable |
| | | | | flowable | | |

### Example 3

Mixtures of Sokalan® PA 40 (=polyacrylate, sodium salt) and in each case 2% by weight and 5% by weight of the hydrophobically modified silicas Sipernat® D 10 and Sipernat® D 17, respectively, were prepared. To this end, the requisite amounts of the two components were gently mixed in a container for 2 min using a tumble mixer. To investigate storage stability, the mixtures were stored in a Petri dish in a conditioning cabinet at 33° C./65% relative humidity and visually assessed for 7 days. For comparison, Sokalan® PA 40 was stored without added silicas.

TABLE 3
storage test of Sokalan ® PA 40 (=modified polyacrylate, sodium salt) with and
without addition of Sipernat ® D 10 or Sipernat ® D 17. Visual assessment at
33° C./65% relative humidity.

| | Visual assessment at 33° C./65% relative humidity | | | | | |
|---|---|---|---|---|---|---|
| Sokalan ® PA 40 | 0 h | 4 h | 8 h | 24 h | 3 days | 7 days |
| a) no additive | flowable | substantially | about | completely | — | — |
| | powder | adhered | 50% run | run away in | | |
| | | at surface | away in | paste form | | |
| | | | paste form | | | |
| b) with 2% of | very | very | readily | powder | powder | powder |
| Sipernat ® D 10 | readily | readily | flowable | swollen but | swollen | swollen |
| | flowable | flowable | powder | flowable | but | but |
| | powder | powder | | | flowable | flowable |
| c) with 5% of | very | very | very | flowable | flowable | powder |
| Sipernat ® D 10 | readily | readily | readily | powder | powder | swollen |
| | flowable | flowable | flowable | | | but |
| | powder | powder | powder | | | flowable |
| d) with 2% of | very | very | very | powder | powder | powder |
| Sipernat ® D 17 | readily | readily | readily | swollen but | swollen | swollen |
| | flowable | flowable | flowable | flowable | but | but |
| | powder | powder | powder | | flowable | flowable |
| e) with 5% of | very | very | very | flowable | flowable | powder |
| Sipernat ® D 17 | readily | readily | readily | powder | powder | swollen |
| | flowable | flowable | flowable | | | but |
| | powder | powder | powder | | | flowable |

### Example 4

Mixtures of Sokalan® PA 40 (=polyacrylate, sodium salt) and in each case 2% by weight and 5% by weight of the hydrophobically modified silicas Sipernat® D 10 and Sipernat® D 17, respectively, were prepared. To this end, the requisite amounts of the two components were gently mixed in a container for 2 min using a tumble mixer. To investigate storage stability, the mixtures were stored in a Petri dish in a conditioning cabinet at 38° C./78% relative humidity and visually assessed for 32 hours. For comparison, Sokalan® PA 40 was stored without added silicas.

TABLE 4
storage test of Sokalan ® PA 40 (= modified polyacrylate, sodium salt) with and without
addition of Sipernat ® D 10 or Sipernat ® D 17. Visual assessment at
38° C./78% relative humidity.

| | Visual assessment at 30° C./65% relative humidity | | | | |
|---|---|---|---|---|---|
| Sokalan ® PA 40 | 0 h | 4 h | 8 h | 24 h | 32 h |
| a) no additive | flowable | substantially | about | completely | — |
| | powder | adhered | 50% run | run away in | |
| | | at surface | away in | paste form | |
| | | | paste form | | |
| b) with 2% of Sipernat ® | very | very | readily | powder | powder |
| D 10 | readily | readily | flowable | swollen but | swollen |
| | flowable | flowable | powder | flowable | but |
| | powder | powder | | | flowable |
| c) with 5% of Sipernat ® | very | very | very | flowable | flowable |
| D 10 | readily | readily | readily | powder | powder |
| | flowable | flowable | flowable | | |
| | powder | powder | powder | | |
| d) with 2% of Sipernat ® | very | very | very | powder | powder |
| D 17 | readily | readily | readily | swollen but | swollen |
| | flowable | flowable | flowable | flowable | but |
| | powder | powder | powder | | flowable |
| e) with 5% of Sipernat ® | very | very | very | flowable | flowable |
| D 17 | readily | readily | readily | powder | powder |
| | flowable | flowable | flowable | | |
| | powder | powder | powder | | |

### LIST OF TRADENAMES USED

| | | |
|---|---|---|
| Sokalan ® PA 40 Pulver | (BASF) | polyacrylic acid, sodium salt, |
| | | powder, 15 000 g/mol, |
| | | 93% pure |
| Sokalan ® CP 42 Granulat | (BASF) | modified polyacrylic acid, |
| | | sodium salt, granules, 97% pure |
| Sokalan ® CP 50 Granulat | (BASF) | modified polyacrylic acid, |
| | | sodium salt, granules, 97% pure |
| Sipernat ® D 10 | (Evonik) | hydrophobically modified |
| | | precipitated silica |
| Sipernat ® D 17 | (Evonik) | hydrophobically modified |
| | | precipitated silica |

1-9. (canceled)

10. A mixture comprising powder-shaped carboxyl-containing polymer, granule-shaped carboxyl-containing polymer or pellet-shaped carboxyl-containing polymer and finely divided additive.

11. The mixture according to claim 10, wherein the carboxyl-containing polymer is selected from the group consisting of homo-polymers of acrylic acid, copolymers of acrylic acid, methacrylic acid, maleic acid, fumaric acid, itaconic acid, citraconic acid, mesaconic acid, aconitic acid and/or methylenemalonic acid and optionally their esters; their amides; C2- to C18-olefins; vinyl acetate; vinyl alcohol; styrene; vinyl methyl ether; vinylcyclohexane; vinyl chloride; acrylonitrile; N-vinylimidazoline, N-vinylpyrrolidone, N-vinylpyridine, diallyldimethylammonium chloride, N-vinylformamide, N-vinylacetamide, vinylamine and allylamine.

12. The mixture according to claim 10, wherein the carboxyl-containing polymer is selected from the group of consisting if homo-polymers of acrylic acid, copolymers of acrylic acid, methacrylic acid, maleic acid, fumaric acid, itaconic acid, citraconic acid, mesaconic acid, aconitic acid and/or methylenemalonic acid and optionally their esters; ethylene, propylene, butylene, diisobutene; vinyl acetate; vinyl alcohol; styrene; vinyl methyl ether; vinylcyclohexane; vinyl chloride; acrylonitrile; N-vinylimidazoline, N-vinylpyrrolidone, N-vinylpyridine, diallyldimethylammonium chloride, N-vinylformamide, N-vinylacetamide, vinylamine and allylamine.

13. The mixture according to claim 10, consisting of 90.0%-99.99% by weight of a carboxyl-containing polymer and 0.01%-10.0% by weight of an additive.

14. The mixture according to claim 10, wherein the additive is a silica having a specific surface area of 30-500 m²/g.

15. The mixture according to claim 10, wherein the additive is a silica having a tamped density of 50-300 g/L.

16. The mixture according to claim 10, wherein the additive is the Ca²⁺ or Mg²⁺ salt of a C₁₆-C₂₂ fatty acid.

17. The mixture according to claim 13, wherein the additive is a silica having a tamped density of 50-300 g/L.

18. The mixture according to claim 13, wherein the additive is the Ca²⁺ or Mg²⁺ salt of a C₁₆-C₂₂ fatty acid.

19. A process for the manufacture of laundry detergents or cleaners which comprises utilizing the mixture according to claim 10.

20. A laundry detergent or cleaner which comprises utilizing the mixture according to claim 10.

21. A textile auxiliary, a fermentation process, a metal processing, a drill oil, a drawing oil emulsion, a water-thinnable and water-soluble metal processing auxiliary, a hydraulic solution and emulsion and to avoid the precipitation of calcium salts of fatty acids and their dispersal in aqueous cooling lubricant; a grinding auxiliary for the comminution of solids, a manufacture of printing inks; a stabilize crop protection agent dispersion; a protective colloid in rubber production, a process for coal-water slurry, a dispersant for a filler; a stabilize and superplasticize ceramic slip; a process for mining of ores for floatation concentration in ore beneficiation; a surface technology, a process to improve the stability of electrocoating baths and to optimize the reactions taking place at the anode and the cathode; a textile finishing in processing and finishing operations during the scouring, kiering, bleaching, dyeing, washing and resin finishing of fabrics and a constituent of a sizing agent; a photographic bath, a developer bath, a bleaching bath or a bleach-fixing bath which comprises the mixture according to claim 10.
